(19) United States
(12) Patent Application Publication
(10) Pub. No.: US 2007/0233238 A1
(43) Pub. Date: Oct. 4, 2007

(54) Devices for Imaging and Navigation During Minimally Invasive Non-Bypass Cardiac Procedures

(75) Inventors: Rany Huynh, Charlestown, MA (US); Nasser Rafiee, Andover, MA (US); Nareak Douk, Lowell, MA (US); Morgan House, Newfields, NH (US); Alex Hill, Minneapolis, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/693,826

(22) Filed: Mar. 30, 2007

Related U.S. Application Data
(60) Provisional application No. 60/744,074, filed on Mar. 31, 2006.
(60) Provisional application No. 60/791,340, filed on Apr. 12, 2006.
(60) Provisional application No. 60/791,553, filed on Apr. 12, 2006.
(60) Provisional application No. 60/793,879, filed on Apr. 21, 2006.

(51) Int. Cl. A61F 2/24; A61B 5/05

(52) U.S. Cl. 623/2.11; 623/2.36; 600/424

(57) ABSTRACT

Delivery devices for placement of therapeutic devices relative a heart valve annulus of a beating heart that are delivered to the heart via minimally invasive surgical procedures and can be used in the heart during a therapeutic procedure that can be visualized in real time and be guided to specific locations within the heart. The systems and methods can be used to determine the exact location of the implantation delivery devices and therapeutic devices relative to a valve annulus and to determine that any therapeutic device is implanted in the correct location.

### CROSS-REFERENCE TO RELATED APPLICATION

The present application claims priority to U.S. Provisional Application No. 60/744,074, filed Mar. 31, 2006 and titled “Devices Having Electromagnetic Coils for Imaging and Navigation During Minimally Invasive Non-Bypass Cardiac Procedures”; U.S. Provisional Application No. 60/791,340, filed Apr. 12, 2006 and titled “Minimally Invasive Procedure for Implanting an Annuloplasty Device”; U.S. Provisional Application 60/791,553, filed Apr. 12, 2006 and titled “Annuloplasty Device Having Helical Anchor Members”; and U.S. Provisional Application 60/793,879, filed Apr. 21, 2006 and titled “Annuloplasty Device Having Helical Anchor Members”, the entire contents of which are incorporated herein by reference in their entireties.

### TECHNICAL FIELD

The invention relates generally to medical devices and particularly to devices, systems, and methods for placing a device in a heart using imaging and navigation during minimally invasive non-bypass procedures.

### BACKGROUND

Heart valves, such as the mitral and tricuspid valves, consist of leaflets attached to a fibrous ring or annulus. These valves are sometimes damaged by diseases or by aging, which can cause problems with the proper functioning of the valve. Referring particularly to the mitral valve, the two native mitral valve leaflets of a healthy heart coapt during contraction of the left ventricle, or systole, and prevent blood from flowing back into the left atrium. However, the mitral valve annulus may become distended for a variety of reasons, causing the leaflets to remain partially open during ventricular contraction and thus allowing regurgitation of blood into the left atrium. This results in reduced ejection volume from the left ventricle, causing the left ventricle to compensate with a larger stroke volume. The increased workload eventually results in hypertrophy and dilatation of the left ventricle, further enlarging and distorting the shape of the mitral valve. If left untreated, the condition may result in cardiac insufficiency, ventricular failure, and possibly even death.

A common procedure for repairing the mitral valve involves implanting an annuloplasty ring on the atrial surface of the mitral valve annulus. During implantation, the annuloplasty ring is aligned with the valve annulus and then fixedly attached to the valve annulus, typically using a suturing process. The annuloplasty ring generally has a smaller internal area than the distended valve annulus so that when it is attached to the annulus, the annuloplasty ring draws the annulus into a smaller configuration. In this way, the mitral valve leaflets are brought closer together, which provides improved valve closure during systole.

Implanting an annuloplasty ring on a valve annulus can be accomplished using a variety of repair procedures, such as procedures that require indirect visualization techniques to determine the exact location of the heart valve and annuloplasty ring during placement of the ring at the valve annulus. Indirect visualization techniques, as described herein, are techniques that can be used for viewing an indirect image of body tissues and/or devices within a patient. One example of such a technique is referred to as endoscopic visualization, which involves displaying images from endoscopic light guides and cameras within the thoracic cavity on a video monitor that is viewed by a surgeon. Effective use of this method depends on having sufficient open space within the working area of the patient's body to allow the surgeon to recognize the anatomical location and identity of the structures viewed on the video display, which can be difficult to accomplish in certain areas of the heart.

Another indirect visualization technique involves the use of fluoroscopy, which is an imaging technique commonly used by physicians to obtain real-time images of the internal structures of a patient through the use of a fluoroscope. However, some tissues, such as the cardiac tissues, do not readily appear under fluoroscopy, making it very difficult to accurately align the annuloplasty ring prior to its implantation. To improve the visualization of the area of interest, radiopaque contrast dye can be used with x-ray imaging equipment. However, when treating the mitral valve, for example, repeated injections of contrast dye are not practical because of rapid wash-out of the dye in this area of high fluid flow. Additionally, to make high-volume contrast injections of this kind, an annuloplasty catheter system would require multiple lumens, undesirably large lumens, and/or an additional catheter, none of which is desirable during catheterization procedures. Furthermore, multiple high-volume contrast injections are not desirable for the patient due to potential complications in the renal system, where the radiopaque contrast medium is filtered from the blood.

A wide variety of other techniques are available for viewing images of cardiac structures, including ultrasonography such as trans-thoracic echocardiography (TTE), trans-esophageal echocardiography (TEE), cardiac magnetic resonance (CMR) including magnetic resonance imaging (MRI) or magnetic resonance angiography (MRA), and computed tomography (CT) including computed tomography angiography (CTA). However, none of the above techniques, used alone or in combination with other available techniques, provides adequate visualization and guidance during catheter-based valve repair procedures.

Annuloplasty procedures can be further complicated by the structure of the valve annulus and the fact that the annulus can undergo significant movement during procedures performed on a beating heart. Since annuloplasty is performed on a beating heart, care must be taken during both systole and diastole when positioning an annuloplasty ring for fixation. With particular reference again to the mitral valve, the mitral valve leaflets are basically flaps or appurtenances attached to the cardiac muscle tissue, creating a pseudo-annulus. In particular, when the mitral valve is closed during systole, a relatively flat floor of the left atrium is formed; however, during diastole, the mitral valve leaflets open towards the ventricular walls such that, in many cases, the valve annulus is not well defined. That is, the mitral valve annulus lacks a definable shelf or ledge for conveniently locating an annuloplasty ring. Without the direct optical visualization that is provided during surgery, it can be difficult to position an annuloplasty ring in abutment with the superior surface of this poorly defined valve annulus. As a result, an annuloplasty ring may be inadvertently affixed in a misaligned position below, above or angled across the valve annulus when using the non-optical imaging techniques of a catheter-based procedure. Affixing the annuloplasty ring in such a misaligned position could have negative consequences for the patient, such as increasing mitral regurgitation and/or triggering ectopic heart beats.

One possible method for mapping the mitral valve annulus and obtaining real time imaging during beating heart surgery is through the use of electromagnetic (EM) imaging and navigation. With EM navigation, a patient is generally placed on a table having a plurality of sensors either on the surface of the table or at positions around the table. The sensors are connected to a processor and the processor knows the positions of the sensors relative to the table. A patient is then placed on the table and immobilized either by anesthesia, restraints, or both. An elongated flexible device having at least three EM coils spaced along its distal portion can then be inserted into the patient's body (into the vascular system for example). The coils are typically made from extremely small diameter material that can be wound around the outside of the device or wound around an interior layer of the device and then covered with an additional layer of material. A very thin wire (or some other electrically conductive material) communicates from an external AC power source to each of these coils. Alternatively, wireless sensors can be used, which can eliminate the need to provide a wire to communicate with the EM coils.

As the elongated device is moved through the body, the sensors can detect the EM signal that is created by the moving coil. The processor then calculates the position of the coils relative to each sensor. The location of the sensors can be viewed on a display device, and the EM navigation can be combined with other navigation/visualization technologies so that the location of the EM coils in a patient's body can be viewed in real time. Additional sensors may also be incorporated into a system using EM navigation to improve the accuracy of the system, such as temporarily attaching sensors to a patient's body. The relationship between all of the sensors can be used to produce the image of the patient's body on the table. Examples of methods and systems for performing medical procedures using EM navigation and visualization systems for at least part of an overall navigation and visualization system can be found, for example, in U.S. Pat. No. 5,782,765 (Jonkman); U.S. Pat. No. 6,235,038 (Hunter et al.); U.S. Pat. No. 6,546,271 (Resifeld); U.S. Patent Application No. 2001/0011175 (Hunter et al.); U.S. Patent Application No. 2004/0097805, (Verard et al.), and U.S. Patent Application No. 2004/0097806 (Hunter et al.), the entire contents of which are incorporated herein by reference.

Another method for mapping the mitral valve annulus and obtaining real time imaging during beating heart surgery is through the use of electro-potential navigation. Electro-potential (EP) navigation is similar to EM navigation in that there are multiple sensors on or around a surface on which a patient is positioned, and the sensors are in communication with a processing device. When using EP navigation, however, a low frequency electrical field is created around the patient, and the coils on the instrument are connected to a DC energy source such that there is a constant energy signal emitting from the coils. The coils create a disturbance in the electrical field as they move through the field, and location of the instrument in the 3D coordinate space is calculated by determining the location of the disturbance in the energy field relative to the sensors.

While the methods, systems, and devices described above provide for real time imaging of devices during certain types of medical procedures, they do not provide a device that can be used to deliver other devices for treating cardiac valve disease. Therefore, it would be desirable to provide a device, system, and method that can utilize accurate, real time images of a heart valve annulus for the catheter based implantation of a therapeutic heart device or administering a heart repair procedure.

### SUMMARY

In one aspect of the invention, a system is provided that includes delivery devices that are used for placement of therapeutic devices in abutment with a heart valve annulus of a beating heart. The delivery devices are designed to be delivered to the heart via minimally invasive surgical procedures and can be used in the heart during a therapeutic procedure that can be visualized in real time and be guided to specific locations within the heart. An example of such a procedure is repair of a cardiac valve such that the size and shape of the valve annulus must be determined. A more specific example is minimally invasive surgical implantation of a device to treat mitral regurgitation that is performed off bypass on a beating heart. The systems and methods of the invention can be used to determine the exact location of the implantation delivery devices and therapeutic devices relative to the mitral valve annulus and to determine that any therapeutic device used for treating mitral regurgitation is implanted in the correct location.

One aspect of the present invention is a system that comprises delivery devices having an elongated shaft for insertion into a patient's body and a shaped distal portion for implantation of a device for treating heart valve regurgitation. Each of the delivery devices includes at least three EM coils spaced from each other and disposed along the distal portion for EM imaging of the delivery device while it is in a patient's body. The EM coils are connected to an external power source, and the delivery device can be connected to a processor that is part of a larger EM navigation system. Wireless sensors may be used for communication with the EM navigation system. The EM navigation system can comprise at least a plurality of sensors and/or transmitters having a known location relative to a patient, a processor that can be used to determine the location of the EM coils relative to the sensors, a power source, and a display device for viewing the movement, shape, and location of the delivery device in real time.

The delivery devices of the current invention can be delivered to the left atrium via an opening created during a minimally invasive surgical procedure. Once in the atrium, the devices can be viewed in real time while they are used to position and surgically implant a device for treating mitral regurgitation. Examples of devices for treating mitral regurgitation can be found in the following references, which describe the delivery of those devices by catheter, although the disclosed devices can also be made such that they are equally suited for use during minimally invasive surgical procedures: U.S. Patent Application No. 2007/0051377 (Douk et al.); and U.S. Patent Application No. 2007/0027533 (Douk); the contents of which are incorporated herein by reference.

One method of using the current invention involves first mapping and recording the shape of a valve annulus using a specific imaging modality (e.g., magnetic resonance imaging (MRI)), and then registering and importing the information into an EM navigation system. The heart is accessed via minimally invasive surgery, and a delivery device with at least three EM coils is placed through a hole in the left atrium wall. The coils on the distal section of the device are placed adjacent to previously designated navigation points, and a therapeutic device is implanted in the beating heart.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

The present invention will be further explained with reference to the appended Figures, wherein like structure is referred to by like numerals throughout the several views, and wherein:

FIG. 1 is a side view of a tool for delivering a therapeutic device to a heart valve, having EM coils arranged at a distal end of the tool, in accordance with the invention;

FIG. 2 is a side view of one embodiment of a distal end of a delivery device having EM coils, which can be used for delivering a therapeutic device to the anterior side of a mitral valve;

FIG. 3 is a side view of another embodiment of a distal end of a delivery device, which can be used for delivering a therapeutic device to the anterior side of a mitral valve;

FIGS. 4 and 5 are side views of two embodiments of a distal end of a delivery device, both of which are shaped for delivering a therapeutic device to the posterior side of a mitral valve;

FIG. 6 is a partial cutaway view of a heart, illustrating locations for accessing the atrium in accordance with the invention;

FIG. 7 is an enlarged front view of a mitral valve, with a tool of the invention placed on the annulus of the mitral valve on its posterior side;

FIG. 8 is a partial cutaway view of a heart, illustrating the placement of delivery devices for treating cardiac regurgitation according to the invention;

FIG. 9 is an enlarged front view of a mitral valve, showing the placement of a device for treating mitral regurgitation according to the invention; and

FIG. 10 is a block diagram illustrating an EM imaging/navigation system according to the invention.

### DETAILED DESCRIPTION

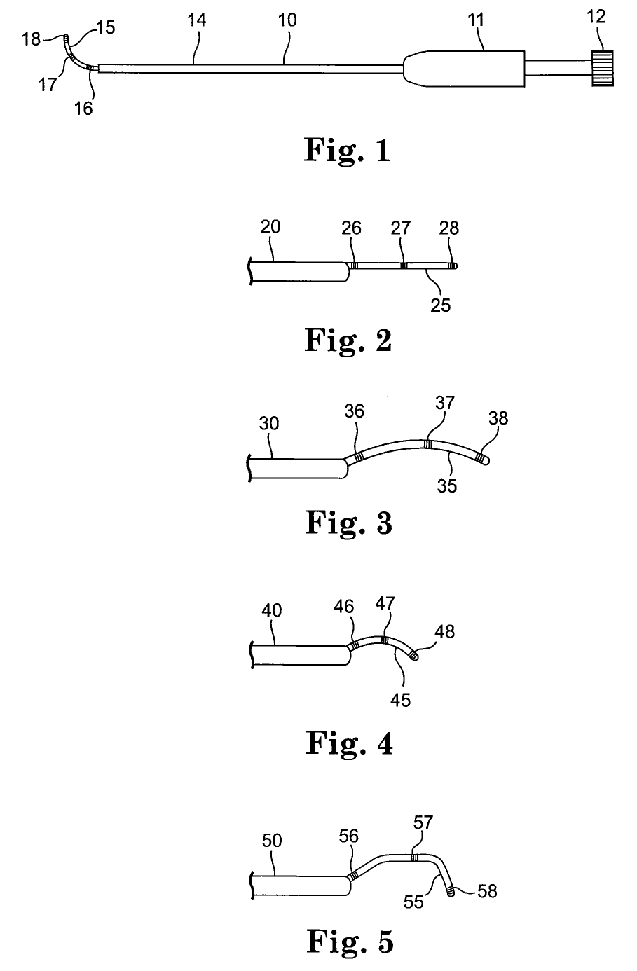

Referring now to the Figures, wherein the components are labeled with like numerals throughout the several Figures, and initially to FIG. 1, one preferred configuration of a delivery device 10 for delivering a device to a predetermined area of the heart for reducing cardiac regurgitation or for treating other heart conditions is illustrated. The delivery device 10 generally comprises a handle 11, a rotatable knob 12 at a proximal end of the device 10, a relatively rigid and elongated shaft 14, and a relatively rigid distal section 15. The knob 12 is connected to an anchor delivery mechanism, and a tether and a helical anchor (not visible) can be disposed in the shaft 14. The helical anchor may be a system or a portion of a system that is referred to herein as a helically anchored device or ring that comprises at least two helical anchor sections and a tether that is routed through the center of the helical anchors to surround a cardiac valve when implanted. When the delivery device 10 is used during an annuloplasty procedure, as will be described in further detail below, the delivery device is positioned in the heart chamber so that its distal section 15 is positioned on the valve annulus. The knob 12 can then be rotated to cause the helical anchor to be translationally rotated out of the shaft to engage with the valve annulus. The helical anchor follows the shape of the distal section 15 of the delivery device 10 as it is rotated out of the elongated shaft. The distal section 15 of the delivery device 10 is kept in contact with the annulus during the procedure to insure that the anchor is correctly implanted.

The terms “distal” and “proximal” are used herein with reference to the treating clinician during the use of the catheter system, where “distal” indicates an apparatus portion distant from, or a direction away from the clinician (e.g., EM coils can be on the “distal” end of the various system members) and “proximal” indicates an apparatus portion near to, or a direction towards the clinician. The delivery devices of the current invention may be made, in whole or in part, from one or more materials that are viewable by radiography, ultrasound, or magnetic resonance imaging visualization techniques. Embodiments of the devices may also be coated with materials that are visible using such visualization methods.

Much of the discussion herein relates to use of the disclosed delivery devices for placement of a heart repair or treatment device in the heart during mitral valve repair procedures. In particular, the delivery devices of the invention, such as delivery device 10, are particularly described as being used for minimally invasive surgical delivery of a cardiac valve annuloplasty ring to a cardiac valve annulus while the heart is beating. However, those with skill in the art will recognize that catheter systems of the invention may also be deployed at other cardiac valves or other locations in the body, and/or may be used to implant devices within the body other than helically anchored devices.

One exemplary method that can be used for accessing a beating heart via minimally invasive surgical procedures generally can start with intubating a patient with a double-lumen endobronchial tube that allows selective ventilation or deflation of the right and left lungs. The left lung is deflated, thereby helping to provide access to the surface of the heart. The patient is rotated approximately 30 degrees with the left side facing upwardly. The left arm is placed below and behind the patient so as not to interfere with tool manipulation during the procedure. While port positions depend to a large extent on heart size and position, in general a seventh and fifth space mid (to posterior) axillary port for tools and a third space anterior axillary port for the scope is preferable. A variety of endoscopes or thoracoscopes may be used including a 30-degree offset viewing scope or a straight ahead viewing scope. In general, short 10 to 12 mm ports are sufficient. Alternatively, a soft 20 mm port with an oval cross section sometimes allows for two tools in the port without compromising patient morbidity.

In one embodiment of the present invention, passages are made through the skin into the thoracic cavity. The passages may be formed by employing one-piece rods or trocars of prescribed diameters and lengths that are advanced through body tissue to form the passage, which are subsequently removed so that other instruments can be advanced through the passage. The passage may instead be formed by employing two-piece trocars that comprise a tubular outer sleeve, which is sometimes referred to as a port or cannula or as the tubular access sleeve itself, having a sleeve access lumen extending between lumen end openings at the sleeve proximal end and sleeve distal end. The two-piece trocar can further include an inner puncture core or rod that fits within the sleeve access lumen. The inner puncture rod typically has a tissue penetrating distal end that extends distally from the sleeve distal end when the inner puncture rod is fitted into the sleeve access lumen for use. The two-piece trocar can be assembled and advanced as a unit through body tissue, and then the inner puncture rod is removed, thereby leaving the tubular access sleeve in place to maintain a fixed diameter passage through the tissue for use by other instruments.

In one embodiment, a tubular access sleeve is placed through a passage that is made as described above in the chest wall of a patient between the patient's second rib and sixth rib, for example. The selection of the exact location of the passage is dependent upon a patient's particular anatomy. A further conventional tubular access sleeve can be placed in a different passage that is also made in the chest wall of patient.

In accordance with one method used in the invention, the patient's left lung is deflated to allow unobstructed observation of the pericardium employing a thoracoscope or other imaging device that is inserted through a sleeve lumen of a tubular access sleeve. The thoracoscope or other imaging device may have its own light source for illuminating the surgical field. Deflation of the patient's lung may be accomplished in a number of ways, such as by inserting a double lumen endotracheal tube into the trachea, and independently ventilating the right, left or both lungs. The left lung can be collapsed for visualization of the structures of the left hemi-sternum when ventilation of the left lung is halted and the left thoracic negative pressure is relieved through a lumen of the tubular access sleeve or a further access sleeve to atmospheric pressure. After deflation, the thoracic cavity may be suffused with a gas (e.g., carbon dioxide) that is introduced through a lumen of the tubular access sleeve or the further access sleeve to pressurize the cavity to keep it open and sterile. The pressurized gas keeps the deflated lung away from the left heart so that the left heart can be viewed and accessed and provides a working space for the manipulation of the tools of the present invention. It will be understood that the access sleeve lumens must be sealed with seals about instruments introduced through the lumens if pressurization is to be maintained.

A thoracoscope can then be inserted into the lumen of a tubular access sleeve to permit wide angle observation of the thoracic cavity by a surgeon directly through an eyepiece or indirectly through incorporation of a miniaturized image capture device (e.g., a digital camera) at the distal end of the thoracoscope or optically coupled to the eyepiece that is in turn coupled to an external video monitor. The thoracoscope may also incorporate a light source for illuminating the cavity with visible light so that the epicardial surface can be visualized. The thoracoscope may be used to directly visualize the thoracic cavity and obtain a left lateral view of the pericardial sac or pericardium over the heart.

The elongated access sleeve provides an access sleeve lumen, enabling introduction of the distal end of a pericardial access tool. The tubular access sleeve and the pericardial access tool are employed to create an incision in the pericardial sac so that the clinician can view and access the left free wall of the heart. After the clinician gains access to the heart, a purse string suture is placed in the free wall of the left atrium (near the commissure of the mitral valve, and above the coronary sinus). The wall is then punctured inside the perimeter of the suture. The wall can be punctured using a special puncture device, or the distal end of the delivery devices described herein can be used to puncture the wall.

The distal end of a first delivery device, such as delivery device 10 of FIG. 1, can then be advanced through the elongated access sleeve, through the puncture formed through the myocardium, and placed against the mitral valve annulus on either the anterior leaflet side (anterior side) or posterior leaflet side (posterior side) of the valve. At least a portion of a device for treating mitral regurgitation can then be implanted. The first delivery device is then withdrawn. The distal end of a second delivery device, which may be generally the same or different from the delivery device 10, is then advanced through the elongated access sleeve, through the puncture formed through the myocardium, and placed against the mitral valve annulus on the other of the anterior or posterior side of the valve. The remainder of the device for treating mitral regurgitation can then be implanted. The second delivery device is then withdrawn and the purse string is tightened to close the puncture. The lung can then be inflated, the instruments withdrawn from the patient, and all openings closed. The procedure outside of the heart can be viewed through a scope as disclosed above, and the procedure in the heart can be visualized and imaged using a number of techniques known in the art. Additionally, EM navigation and imaging can be used to deliver the therapeutic device to a precise location.

As illustrated, the delivery device 10 has three EM coils 16, 17, & 18 spaced along the curved distal section thereof. The EM coils comprise a thin wire made of some biocompatible metal, and the coils preferably have an inductance of over 70 microHenrys (μH). All of the coils of the currently described embodiments can be made from such materials and wrapped around delivery devices a sufficient number of times to have the desired inductance. In one embodiment, the wire is wrapped around the delivery device 25 times, although more or less wrappings can be used. A thin communication wire (not shown) can be embedded in the distal section 15 and the shaft 14 of the delivery device 10, or affixed to the outside of the delivery device 10. The communication wire conducts a charge between the coils 16, 17, 18 and an external AC power source (not shown). Suitable metals for the EM coil and the communication wire include, but are not limited to, copper, silver, gold, platinum and alloys thereof. In one preferred embodiment, the EM coil and the communication wire are both made from copper wires having a diameter of 0.001 inch (0.025 mm). Alternatively, the system may include wireless sensors that do not require the use of such a communication wire associated with the delivery device.

Prior to implanting the helically anchored device or ring, the shape and orientation of the mitral valve annulus can be determined using a separate device as part of an EM navigation and imaging system. The device, which preferably includes at least three EM coils, would be placed on the valve annulus or any corresponding anatomy, such as the coronary sinus, and manipulated to mimic the shape of the annulus so that the clinician could get an accurate image of the size and orientation of the annulus prior to beginning the procedure for implanting a repair device, such as a helically anchored device.

In any case, in preparation for implanting a helically anchored device or ring, a clinician who is mapping and imaging the mitral valve annulus may designate several points for subsequent alignment of EM coils on the delivery devices, such as the helical anchor members. The location of the designated points are preferably selected to that the device or ring can be properly implanted, thereby minimizing the chances of injuring a patient and optimizing the opportunity to reduce mitral regurgitation. During the implantation procedure, the EM coils on the delivery device, such as delivery device 10, are aligned with these pre-designated points to ensure proper alignment of the delivery device before the helical anchors are implanted. In addition to designating points for anchor placement, the clinician can also identify and designate the location along the annulus nearest to the commissure. This piece of information can be used during the procedure as another data point used by the clinician to properly align the puncture device so the heart is punctured at the correct location for insertion of the delivery devices.

Referring again to FIG. 1, the distal section 15 of the delivery device has at least a slight curvature, which is selected to mimic the general shape of a particular valve annulus to which it will be delivering a heart repair or treatment device (e.g., a helical anchor). To make sure that the correct shape and size of delivery tool is used, the clinician can evaluate the size and shape of the valve annulus during a mapping and imaging procedure, such as the procedure described above, which is conducted prior to implanting the heart repair or treatment device. The delivery devices disclosed herein can be configured for use on the anterior side of the mitral valve or the posterior side of the valve, which typically have different curvatures.

For delivery of a device to the anterior side of the valve, FIGS. 2 and 3 each illustrate an exemplary distal section of a delivery device that is particularly shaped for delivering a helical anchor for a helically anchored device or ring to the anterior side of the annulus. The curves in the distal sections 25 and 35 of delivery devices 20 and 30, respectively, will typically be shallower than the curves of similar devices that can be used on the posterior side of the same valve. Delivery device 20 includes three EM coils 26, 27, 28, as described above, which are spaced from each other along the distal portion 25 thereof, and delivery device 30 includes three EM coils 36, 37, 38, which are spaced from each other along the distal portion 35 thereof. In one embodiment of the invention, the delivery device will have the same number of coils (e.g., three EM coils) as the quantity of predesignated locations in the anatomy of the patient, where these predesignated locations can be provided using a number of mapping techniques. However, it is understood that the delivery device may include more or less than the number of predesignated locations in the anatomy of the patient such that either all of the EM coils of the device are not used or such that all of the predesignated locations are not used in a particular placement of a device.

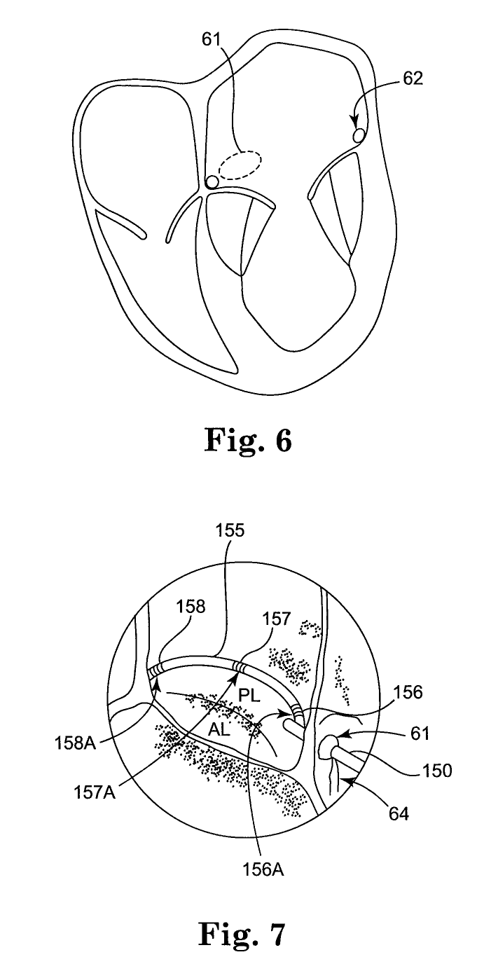

FIG. 4 and FIG. 5 each illustrate an exemplary distal section of a delivery device that is particularly shaped for delivering a helical anchor for a helically anchored device or ring to the posterior side of the annulus. The curves in the distal sections 45 and 55 of delivery devices 40 and 50, respectively, will typically (but not necessarily) be sharper or more pronounced than the curves of the devices for use on the anterior side of the valve. Delivery device 40 includes three EM coils 46, 47, 48, as described above, which are spaced from each other along the distal portion thereof, and delivery device 50 includes three EM coils 56, 57, 58, which are spaced from each other along distal portion 55 thereof. The shapes of the distal sections shown in FIGS. 2 through 5 should not be considered to be all of the possible shapes and sizes available, but are shown herein to exemplify that a plurality of possible shapes and sizes exist for the delivery devices, which are related to the size and shape of a particular valve annulus. It is possible, however, for a certain number of “standard” delivery devices to be provided to a clinician, which devices would encompass a majority of sizes and shapes of annuluses that are typically encountered for that valve (e.g., the mitral valve). In this way, one of these delivery devices can be selected for the implantation process from a group or stock of such delivery devices. In addition, distal sections of other “custom” delivery devices may be particularly designed with a special shape and/or size for a specific patient if the mapping and imaging procedures identify an annulus shaped such that no existing tools will be suitable for use in implanting a particular heart repair or treatment device.

FIGS. 6 and 7 illustrate an exemplary placement of delivery devices of the current invention inside the heart. To access the atrium, a purse string suture is placed in the heart and the wall is punctured (as described above) at a location 61 in the atrium wall at a location adjacent the commissure of the posterior and anterior cusp and above the coronary sinus. The delivery devices can then be placed on the valve annulus 62 and the heart repair or treatment device can then be surgically implanted or otherwise positioned relative to the annulus 62. Referring particularly to FIG. 7, the location of the puncture 61 is visible inside of the purse string suture 64 (the free ends of the which are visible in the figure), and a portion of a delivery device 150 is illustrated for delivering a heart repair or treatment device to the posterior leaflet (PL) side of a mitral valve. Delivery device 150 includes a distal section 155 that is placed against the mitral valve so that its three EM coils 156, 157, 158 are positioned generally adjacent to three designated points 156A, 157A, and 158A. These three designated points 156A, 157A, 158A can be selected and located in a number of ways, including the methods discussed above for mapping the shape and size of the valve annulus. The clinician can view the EM coils 156, 157, 158 of the distal section, in real time, on a display device that is connected to an EM navigation system. The helical anchor or other device or devices can thereby be implanted in the correct location.

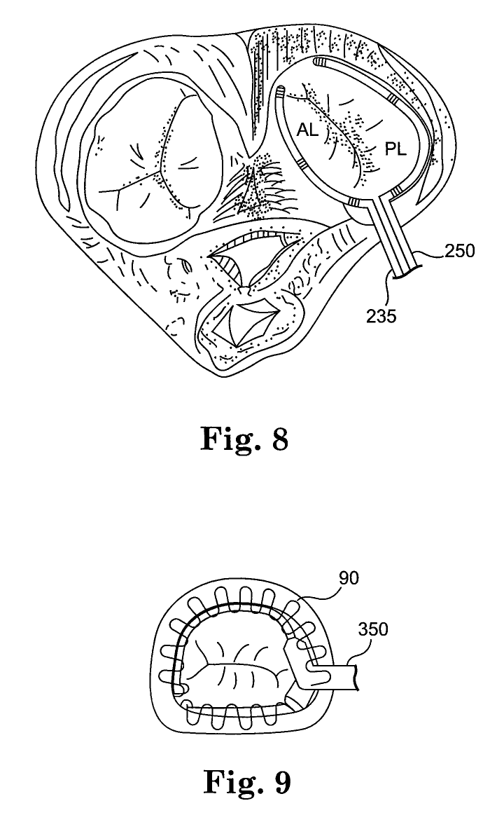

Referring now to FIG. 8, a schematic cross section is illustrated for placing two delivery devices 235, 250 on a valve annulus. In particular, the figure shows how the posterior delivery device 250 and the anterior delivery device 235 are oriented after insertion into the atrium. The distal portions of the devices 235, 250 are sized and shaped for this particular annulus based on the previously performed imaging and mapping. As is represented by the exemplary pronounced curvature of the distal section of the posterior delivery device 250 in this figure, the distal section is relatively rigid so that the heart walls can be shaped to conform to the shape of the valve annulus and the device distal section for implantation of the helical anchor of a helically anchored device or ring.

FIG. 9 shows a representation of a mitral valve as seen from above with a distal portion of a delivery device 350 for implanting a helical anchor 90 in a valve annulus positioned on the posterior side of the valve. Helical anchor sections are implanted into the valve tissue, and a tether (not shown) can be routed through the anchor sections and tightened to improve coaption of the valve leaflets and reduce mitral regurgitation. As illustrated, the helical anchor section comprises an elongate coiled member that may have a tissue penetrating tip at its distal end and a proximal end that is connected to a driver of the delivery system, although other configurations of the heart repair and treatment devices can alternatively be implanted.

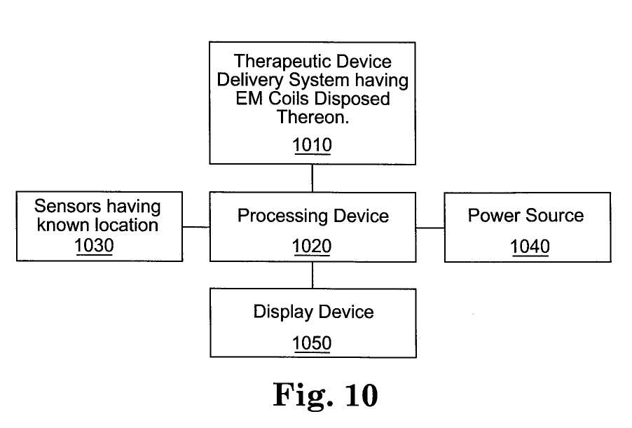

Referring to FIG. 10, a block diagram of a system for delivering a therapeutic device to a valve annulus or other structure within a vascular system is shown. In particular, the system comprises a device delivery system 1010 having a selection of delivery devices for use during minimally invasive procedures as described above, the devices each having at least three EM coils spaced from each other on a distal section thereof. The devices of the delivery system 1010 can be attached to a processing device 1020 and the processing device 1020 is also in signal communication with a plurality of sensors 1030 having a known location, and a display device 1050. A power source 1040 provides power to the processing device 1020, and it can also provide power to each of the other components of the system through the processing device 1020 or separately. In alternate embodiments of the system, each component can have its own separate power source. In another embodiment, the delivery devices of the delivery system are not connected to the processing device.

As discussed above, aspects of the invention include a system for accurately delivering therapeutic devices to a cardiac valve or other vascular structure using EM navigation techniques. While the devices in this disclosure have been discussed in terms of having transmitters on the delivery devices and receivers/sensors outside of a patent's body, this can be reversed such that the sensors are on the delivery devices and the transmitters are outside the body. Alternately, a system could be used where both transmitters and sensors are on the delivery devices, and both transmitters and sensors are located outside of the patent's body.

The currently disclosed delivery devices can also be connected to a DC power source and used in an EP navigation system as described above. The devices and methods disclosed herein can also be used in combination with other visualization/imaging devices and methods to provide a clinician with a detailed understanding of a particular patient's vasculature.

Some embodiments of the devices disclosed herein can include materials having a high X-ray attenuation coefficient (radiopaque materials). The devices may be made in whole or in part from the material, or they may be coated in whole or in part with radiopaque materials. Alloys or plastics may include radiopaque components that are integral to the materials. Examples of suitable radiopaque material include, but are not limited to gold, tungsten, silver, iridium, platinum, barium sulfate and bismuth sub-carbonate.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. A system for delivering a device to a location of an anatomical structure, the system comprising:
a delivery device comprising a plurality of electromagnetic coils spaced from each other along a distal end portion of the delivery device, wherein the delivery device is moveable for positioning each of the plurality of electromagnetic coils relative to each of a plurality of predetermined locations of the anatomical structure; and
a processor for determining the locations of the electromagnetic coils relative to the predetermined locations of the anatomical structure and relative to at least one sensor.

2. The system of claim 1, wherein the anatomical structure is a mitral valve annulus.

3. The system of claim 1, further comprising a navigation system comprising the at least one sensor, the processor, at least one power source, and a display device for viewing the electromagnetic coils of the delivery device during delivery of the device.

4. The system of claim 1, further comprising a device to be implanted relative to the anatomical structure.

5. The system of claim 1, wherein each of the electromagnetic coils has a corresponding predetermined location of the anatomical structure.

6. The system of claim 1, wherein the delivery device comprises at least three electromagnetic coils.

7. The system of claim 1, wherein the delivery device further comprises a first placement component for delivering a therapeutic device to a first side of the anatomical structure, wherein the first placement component comprises a distal end having a first curvature.

8. The system of claim 7, wherein the delivery device further comprises a second placement component for delivering a therapeutic device to a second side of the anatomical structure, wherein the second placement component comprises a distal end having a second curvature that is different from the first curvature.

9. The system of claim 8, wherein the first side of the anatomical structure comprises the anterior side of a mitral valve annulus, and wherein the second side of the anatomical structure comprises the posterior side of a mitral valve annulus.

10. The system of claim 8, wherein the first and second placement components each comprise at least three electromagnetic coils spaced from each other along their respective distal ends.

11. The system of claim 1, wherein the delivery device comprises a single component having a distal end portion for delivering a therapeutic device to the entire anatomical structure.

12. A method of delivering a therapeutic device to a mitral valve annulus, the method comprising the steps of:
providing a delivery device comprising a plurality of electromagnetic coils spaced from each other along a distal end portion of the delivery device;
positioning the delivery device adjacent to the mitral valve annulus so that at least three of the plurality of electromagnetic coils are proximal to at least three corresponding predetermined locations on the anatomical structure; and
delivering the therapeutic device to the mitral valve annulus.

13. The method of claim 12, wherein the therapeutic device is configured to treat mitral valve regurgitation.

14. The method of claim 12, wherein the delivery device further comprises a first placement component for delivering a therapeutic device to an anterior side of the mitral valve annulus, wherein the first placement component comprises a distal end having a first curvature that is preselected to mimic the shape of the anterior side of the mitral valve annulus.

15. The method of claim 14, wherein the delivery device further comprises a second placement component for delivering a therapeutic device to a posterior side of the mitral valve annulus, wherein the second placement component comprises a distal end having a second curvature that is different from the first curvature.

16. The method of claim 15, wherein the first and second placement components each comprise at least three electromagnetic coils spaced from each other along their respective distal ends.

17. The method of claim 15, wherein the first and second placement components are simultaneously positioned adjacent to the mitral valve annulus for delivering the therapeutic device to the anterior and posterior sides of the mitral valve annulus.

18. The method of claim 15, wherein the first and second placement components are sequentially positioned adjacent to the mitral valve annulus for delivering the therapeutic device to both the anterior side and the posterior side of the mitral valve annulus.

19. The method of claim 12, wherein the positioning the delivery device adjacent to the mitral valve annulus is performed off bypass on a beating heart.

20. The method of claim 12, wherein the electromagnetic coils are in communication with an electromagnetic navigation system, the method further comprising the step of wirelessly communicating with the electromagnetic navigation system using wireless sensors.
